(19) United States
(12) Patent Application Publication
(10) Pub. No.: US 2023/0220002 A1
(43) Pub. Date: Jul. 13, 2023

(54) METHOD FOR SEQUENCING A PROTEIN/POLYPEPTIDE USING AEROLYSIN NANOPORES

(71) Applicant: NANJING UNIVERSITY, Jiangsu (CN)

(72) Inventors: Yitao LONG, Jiangsu (CN); Yilun YING, Jiangsu (CN)

(73) Assignee: NANJING UNIVERSITY, Jiangsu (CN)

(21) Appl. No.: 17/915,496

(22) PCT Filed: May 19, 2021
(86) PCT No.: PCT/CN2021/094480

§ 371 Date: Sep. 29, 2022

(30) Foreign Application Priority Data
Apr. 13, 2020 (CN) 202010286336.4
Oct. 28, 2020 (CN) 202011168150.5

(51) Int. Cl. C07K 1/12; C07K 1/113

(52) U.S. Cl. CPC C07K 1/128; C07K 1/1133

(57) ABSTRACT

The present invention provides a method for sequencing a protein/polypeptide based on Aerolysin nanopores to achieve specific discrimination of natural amino acids and post-translational modifications thereof and accurate acquisition of a sequence of a single-molecule protein, the method including the following steps: (1) unfolding of the protein; (2) terminus labeling of protein sequencing; (3) protein charge screening; (4) unfolding of a tertiary structure of the polypeptides; (5) orthogonal identification of amino acids; (6) confined perturbation-assisted identification of amino acids; and (7) single-molecule protein sequencing. The present invention aims at sensitive detection of sequence information about 20 amino acids and establishes an innovative method for accurately determining sequences of the amino acids and post-translational modifications of a single protein molecule.

### BACKGROUND

### Technical Field

The present invention relates to the technical field of biology, and particularly relates to a method for sequencing a protein/polypeptide based on Aerolysin nanopores.

### Description of Related Art

Thousands of different proteins maintain all functions of a cell, and accurate determination of amino acid sequences of proteins in an organism can provide key information for the understanding of the functions of proteins, the biological processes in which they are involved, and the interactions between a protein and the other protein (or other biomolecules). Depending on the direction of transmission of genetic information, protein synthesis includes processes such as DNA transcription, post-transcriptional processing, translation, and post-translational modification. However, one gene may be spliced in multiple mRNA forms during transcription, and the same protein may be post-translationally modified in many forms, so there are great differences between the genotype and phenotype of the protein. It is necessary to directly and accurately determine sequences of the amino acids and post-translational modifications of a protein.

In recent years, with the increasing demand for single-molecule protein detection, protein analysis techniques at the single-molecule level have been rapidly developed in an attempt to decipher the sequence information of a single protein molecule, such as a protein fingerprinting fluorescence technique and a tunneling current protein detection technique. However, current fluorescence sequencing methods lack efficient organic fluorophores for detecting 20 different amino acids without significant overlap between emission peaks to specifically label the 20 amino acids. In addition, the sub-nanometer scale tunneling measurement interface used in the tunneling current detection technique is difficult to stably prepare. Limited by those challenges, although it is possible to distinguish several amino acids in the prior art, the effective identification of 20 amino acids and their post-translational modifications is still not achieved, let alone the acquisition of sequence information about the amino acids. Therefore, the single-molecule protein sequencing is still facing great challenges at present, and there is an urgent need to develop a new principle for sensitive detection of sequence information about 20 amino acids, and to establish an innovative method for accurately determining sequences of the amino acids and post-translational modifications of a single protein molecule.

### SUMMARY

The technical problems to be solved by the present invention are as follows: the single-molecule protein sequencing is still facing great challenges at present, and there is an urgent need to develop a new principle for sensitive detection of sequence information about 20 amino acids, and to establish an innovative method for accurately determining sequences of the amino acids and post-translational modifications of a single protein molecule.

In order to solve the above technical problems, the present invention provides a method for sequencing a protein/polypeptide based on Aerolysin nanopores to achieve specific discrimination of natural amino acids and post-translational modifications thereof and accurate acquisition of a sequence of a single-molecule protein, the method including the following steps: (1) unfolding of the protein; (2) terminus labeling of protein sequencing; (3) protein charge screening; (4) unfolding of a tertiary structure of the polypeptides; (5) orthogonal identification of amino acids; (6) confined perturbation-assisted identification of amino acids; and (7) single-molecule protein sequencing.

In the unfolding of the protein of step (1), a single protein molecule must have its high order structure unraveled to enter a single nanopore in a linear chain form before the sequencing based on nanopores, and the single protein will be unfolded by a temperature and pH regulation method.

In the terminus labeling of protein sequencing of step (2), the N-terminus or C-terminus of an unfolded polypeptide chain is labeled with a peptide nucleic acid, an oligonucleotide, a polypeptide chain or an organic functional group having a specific sequence as a sequencing origin to obtain an starting label signal of ion flow.

In the protein charge screening of step (3), protein chargeability screening nanopores are designed.

In the unfolding of a tertiary structure of the polypeptides of step (4), a tertiary structure unfolding nanopore is further designed for assisting the chargeability screening, that is, an unfolding region is constructed at the inlet of Aerolysin nanopore to further open a molecular structure of the polypeptide, i.e., mutant T284/F/Y/I/L/W or G214/F/Y/I/L/W.

In the orthogonal identification of amino acids of step (5), aiming at sequencing of linear polypeptide molecules with each chargeability, nanopores at least including the following six types of orthogonal identifications are designed:
(a) identification of a first class of amino acids, including but not limited to H, R, K, E, D, Q, N and W, intended to be achieved based on an electrostatic interaction, i.e., mutant T218K/R/H/D/E, S278K/R/H/D/E, S276K/R/H/D/E, T274K/R/H/D/E or A224QN/D/E/R/K/H;
(b) identification of a second class of amino acids, including but not limited to Q, N, Y, T, S, C, G and H, intended to be achieved based on hydrogen bond and hydrophilic interactions, i.e., mutant T218N/Q, Q212R/K/H, D209S/T, S276Q/N, D222G/A/S or A224E/D, wherein the histidine His has an R group with pKa of 7, and can be made uncharged through fine adjustment of pH, thus enabling differentiation in a specific nanopore based on its hydrogen bond interaction with a key region;
(c) identification of a third class of amino acids, including but not limited to I, L, M, V, P, A, C and G, intended to be achieved based on a van der Waals interaction, i.e., mutant R220S/T/A, D222G/A, S236I/L/V, G270I/L, T232I/L/V, T274G/A/I/L or K238F/Y/W;
(d) identification of a fourth class of amino acids, including but not limited to W, P, F, Y, H, I, L and V, intended to be achieved based on a large p bond in side chains of part of the amino acids, i.e., mutant D222W/H/F/Y, S276F/Y, A224K/R/W, S272W/H or T274W/H/F/Y;
(e) identification of a fifth class of large-volume amino acids, including but not limited to A, C, G, S, T and V, intended to be achieved based on a small steric hindrance effect, i.e., mutant S276F/Y/I/L, S278F/Y/I/L/P, T274W/P, S236W or K238G/W/I/L/F/Y/P; and
(f) identification of a sixth class of small-volume amino acids, including but not limited to W, H, I, K, R and Y, intended to be achieved based on a large steric hindrance effect, i.e., mutant T218G/A, S276G/A, S278G/A, T274G/A, N226D/E or Q268S/T/G/A.

In the confined perturbation-assisted identification of amino acids of step (6), in view of identification errors possibly introduced between part of amino acids with small structural differences and isomer amino acids, alternating electric field and optical perturbation measurement systems are introduced and perturbation amplification nanopores for the perturbation systems are designed to further improve sequencing accuracy in combination with a specific nanopore, wherein the specific nanopore is shown as follows:
(a) mutant S236D/E/K/H/R, A260D/E/K/H/R, K238H/R/D/E, T240D/E or S256H/R/W in combination with the alternating electric field perturbation system; and
(b) mutant S236W/H, K238I/L, S256Y/F/W, P249W or V250I/L/F/Y/W in combination with the optical perturbation system.

The method for the single-molecule protein sequencing of step (7) is performed by:
(a) bringing the protein or polypeptide into a contact with the pore, so that the protein or polypeptide moves relative to the pore; and
(b) measuring the ion current passing through the pore as the protein or polypeptide moves relative to the pore, wherein the current is indicative of one or more characteristics of the protein or polypeptide, and includes shape, amplitude and duration of a current signal, resolving characteristics of the current signal according to a mathematical transformation, and creating a database of polypeptides for mutual correction of data, thereby characterizing the protein or polypeptide.

Preferably, the method for sequencing a protein/polypeptide using Aerolysin nanopores includes the following specific steps:
(1) sample pretreatment: breaking internal hydrogen bonds of the protein by raising the temperature to 60-100° C. and decreasing the pH of a solution to 0-5, and breaking S—S bonds of the protein using a reducing agent tri(2-carboxyethyl)phosphine (TCEP) or dithiothreitol (DTT) at the same time, so that polypeptide chains in the single protein are released and linearized;
(2) specifically modifying the N-terminus of the polypeptide chain with a peptide nucleic acid PNA, an oligonucleotide, a polypeptide chain or an organic functional group, so that a specific ion flow blocked signal or fluorescent signal is generated at the beginning or the end of the polypeptide entering the nanopore, thereby determining the starting point of sequencing of the single polypeptide molecule in the nanopore, and providing a starting time label for mutual correction of parallel sequencing signals of a plurality of orthogonal identification nanopores;
(3) using a denaturant and designing and constructing a “tertiary structure unfolding nanopore” to achieve the unfolding of the tertiary structure of the polypeptide, wherein the “tertiary structure unfolding nanopore” is designed as follows: bionically constructing a central amino acid environment of a proteasome 19S domain at the inlet of the Aerolysin nanopore to enhance a specific non-covalent interaction between the polypeptide and the inlet of the nanopore, and gradually destroying weak interactions inside the polypeptide molecule by virtue of electric driving forces to drive the polypeptide molecule to enter a confined pore and achieve linear unfolding, so that the great challenge of the tertiary structure of the polypeptide on sequencing of the polypeptide in the nanopore is overcome;
(4) designing functionalized Aerolysin nanopores capable of driving polypeptides with different chargeabilities, and preliminarily screening the chargeabilities of the polypeptides to match the selection of orthogonal sequencing nanopores in the next step;
(5) constructing 6 types of orthogonal identification nanopores for specifically identifying amino acid sequences of polypeptides for each chargeability based on an electrostatic interaction, hydrogen bond and hydrophilic interactions, a van der Waals interaction, a large p-bond interaction of amino acids, a large steric hindrance effect and a small steric hindrance effect;
(6) introducing amino acids which are easy to form a hydrogen bond into the inlet region of each orthogonal identification nanopore, and adjusting the confined pore structure of the region, thereby designing and constructing a secondary structure label region of polypeptides, in which amino acid residues inside the pore will have a hydrogen bond interaction with the polypeptides with different secondary structures, thereby inducing changes in specific ion flow blocking and specific ion mobility, and forming ion flow characteristics of the secondary structure label for calibrating and denoising an ion flow electrical signal of single protein sequencing during data processing;
(7) in view of amino acid identification errors possibly existing in the orthogonal amino acid identification, further identifying ion migration frequency characteristics by adopting an ion flow confined perturbation technique in combination with influences of specifically designed amplification temperature perturbation, alternating electric field perturbation and optical perturbation of the nanopore on the ion mobility in the pore, thereby improving the amino acid identification capability at a nanopore measurement interface, and accurately obtaining sequence information of the single protein molecule; and
(8) making one or more measurements as the protein or polypeptide moves relative to the Aerolysin nanopore, specifically, measuring and analyzing a current passing through the pore, including characteristics such as amplitude, frequency, shape and duration of the current, thereby determining the presence or absence of one or more of the characteristics in the analyte; and resolving characteristics of the current signal according to a mathematical transformation, and creating a database of polypeptides for mutual correction of data, thereby characterizing the protein or polypeptide.

Compared with the prior art, the present invention has the following beneficial effects.
(1) Since a high-order structure of a protein, which is formed with a plurality of polypeptide chains coiled and folded via hydrogen bonds or S—S bonds, has large volume, it is difficult for the protein to enter an Aerolysin nanopore which is only 1 nm at its narrowest part. To this end, a protein high-order structure unfolding module is designed to unfold the protein molecule with the high-order structure into linear polypeptide molecules. Based on this, in the present invention, internal hydrogen bonds of the protein are broken by raising the temperature and decreasing the pH of a solution and other ways, and S—S bonds of the protein are broken using a reducing agent such as tri(2-carboxyethyl)phosphine (TCEP) and dithiothreitol (DTT) at the same time, so that a plurality of polypeptide chains in the single protein are released and linearized.

(2) A polypeptide molecule may enter the nanopore starting from the N-terminus or the C-terminus. If an amino acid sequence is read according to a characteristic signal of a timing sequence nanopore, it is necessary to determine the terminus of a single polypeptide molecule as it enters the nanopore, i.e., determine the starting direction of sequencing. In this case, in the present invention, the N-terminus of the polypeptide is specifically modified with a peptide nucleic acid PNA (such as a PNA sequence containing a plurality of adenines), an oligonucleotide, a polypeptide chain or an organic functional group (such as FAM), so that a special signal such as a specific ion flow blocked signal or fluorescence signal is generated at the beginning or the end of the polypeptide entering the nanopore, thereby determining the starting point of sequencing of the single polypeptide molecule in the nanopore, and providing a starting time label for mutual correction of parallel sequencing signals of a plurality of orthogonal identification nanopores.

(3) Since the secondary structure of the polypeptide may be further coiled and folded into a tertiary structure in the solution, enabling the polypeptide to have a large three-dimensional size, it is difficult for the polypeptide to enter the nanopore. In this case, in the present invention, a denaturant (such as guanidine hydrochloride (GdHCl)) is used, and a “tertiary structure unfolding nanopore” is designed and constructed to achieve unfolding of the tertiary structure of the polypeptide. The “tertiary structure unfolding nanopore” is designed as follows: bionically constructing a central amino acid environment (such as mutant T210Y and S213W) of a proteasome 19S domain at an inlet of the Aerolysin nanopore to enhance a specific non-covalent interaction between the inlet of the nanopore and the polypeptide, and gradually destroying weak interactions inside the polypeptide molecule by virtue of various electric driving forces such as an electrophoretic force, an electroosmotic flow, and a dielectrophoretic force to drive the polypeptide molecule to enter a confined pore and achieve the linear unfolding, so that the great challenge of the tertiary structure of the polypeptide on sequencing of the polypeptide in the nanopore is overcome.

(4) In the present invention, functionalized Aerolysin nanopores capable of driving polypeptides with different chargeabilities are designed, and the chargeabilities of the polypeptides are preliminarily screened to match the selection of orthogonal sequencing nanopores in the next step. In the present invention, at least 4 “protein charge screening nanopores” for specifically capturing 4 types of polypeptides with chargeabilities are designed, namely negatively charged polypeptides, positively charged polypeptides, electrically neutral polypeptides with positive and negative charges shielded from each other, and electrically neutral polypeptides with positive and negative charges separated, respectively. The 4 “protein charge screening nanopores” are designed as follows.

(i) A “protein charge screening nanopore” for specifically identifying the negatively charged polypeptides. By adjusting the diameter of the key region inside the pore or transferring charges in the pore to a region of a larger diameter (such as mutant T274N/Q/I/L, T232D/E, K238H/D/R/F/A/C/G/Q/E/K/L/M/N/S/Y/T/I/W/P/V, and S280T/N/Q/H/I/L), the electroosmotic flow inside the pore that is determined by the charge at the narrowest part of the pore is controlled to be zero, and the dielectrophoresis force is reduced, so that a single negatively charged polypeptide is driven into the nanopore by the electrophoresis force.

(ii) A “protein charge screening nanopore” for specifically identifying the positively charged polypeptides. By introducing or increasing the distribution of negative charges in the pore (such as mutant T274D/E, T218D/E, S276D/E, S278D/E, K238A/N/D/E/Q, R282D/E/S/T/N/Q/A, and R220D/E/S/T/N/Q/A), the electroosmotic flow determined by cations inside the pore is adjusted. In the experiment, a reverse voltage is applied to achieve efficient capture of a single positively charged polypeptide.

(iii) A “protein charge screening nanopore” for specifically identifying the electrically neutral polypeptides with positive and negative charges shielded from each other. For the electrically neutral polypeptides with positive and negative charges shielded from each other (that is, the positive and negative charges are relatively close together), by introducing positively charged amino acids into a region of a smaller diameter of the pore (such as mutant T218K/R/H/N/Q, S276K/R/H, S278K/R/H/N/Q, S274K/R/H, N226K/R/H, S272K/R/H, G270K/R/H, S228K/R/H, Q268K/R/H, T230K/R/H, A266K/R/H, T232K/R/H, S264K/R/H, G234K/R/H, N262K/R/H, S236K/R/H, A260K/R/H and S280N/Q), the electroosmotic flow determined by anions inside the pore is enhanced, so that the capture efficiency of the nanopore to the electrically neutral polypeptide with positive and negative charges shielded from each other is enhanced, and a specific ion flow response is obtained.

(iv) A “protein charge screening nanopore” for specifically identifying the electrically neutral polypeptides with positive and negative charges separated. For the electrically neutral polypeptides with positive and negative charges separated, by enhancing the potential gradient at the inlet of the pore (such as mutant S280Q/N/A, T284Q/N/A, and G214Q/N/A), the non-linear electric field strength is adjusted, so that a single electrically neutral polypeptide with positive and negative charges separated is driven into the pore by the dielectrophoretic force.

(5) In the present invention, based on an electrostatic interaction, hydrogen bond and hydrophilic interactions, a van der Waals interaction, a large p-bond interaction of amino acids, a large steric hindrance effect and a small steric hindrance effect, at least 6 types of orthogonal identification nanopores for specifically identifying amino acid sequences are constructed for polypeptide with each chargeability, which are shown as follows.

(I) Based on the electrostatic interaction, charged amino acids are introduced into an existing current sensing region in the pore, such as mutant T218K/R/H/D/E, S278K/R/H/D/E, S276K/R/H/D/E, T274K/R/H/D/E, and A224Q/N/D/E/R/K/H. Meanwhile, the introduction of the charged amino acids can enhance hydrogen bond, salt bond and cation-p interactions between the pore and the amino acids to be sequenced, so that identification of a first class of amino acids, including but not limited to H, R, K, E, D, Q, N and W, is achieved.

(II) Based on the hydrogen bond and hydrophilic interactions, the potential gradient of a current sensing region in the pore is regulated, such as mutant T218N/Q, Q212R/K/H, D209S/T, S276Q/N, D222G/A/S, and A224E/D, to increase the speed of charged amino acids passing through the region and prolong the retention time of polar uncharged amino acids in the region, so that identification of a second class of amino acids, including but not limited to Q, N, Y, T, S, C, G, and H, is achieved. The histidine H has an R group with pKa of about 7, and can be made uncharged through fine adjustment of pH, thus enabling characteristic differentiation in a specific nanopore based on its hydrogen bond interaction with a key sensing region of the nanopore.

(III) Based on the van der Waals interaction, the overall potential distribution and the stereostructure distribution of the pore are regulated, and hydrophobic amino acids are introduced, such as mutant R220S/T/A, D222G/A, S236I/L/V, G270I/L, T232I/L/V, T274G/A/I/L and K238F/Y/W, to transfer the current sensing region from a electrostatic sensitive region of a wild-type pore to a hydrophobic region of a mutant pore, and prolong the retention time of a specific amino acid in the region through the interaction to obtain a characteristic ion flow signal, so that identification of a third class of amino acids, including but not limited to I, L, M, V, P, A, C and G, is achieved.

(IV) Based on the large p-bond interaction of part of amino acids, the composition of a current sensing region in the Aerolysin nanopore is reconstituted on the basis of regulating the stereostructure and potential distribution of the pore, and a sensitive region with positively charged amino acids and hydrophobic amino acids predominated is constructed, such as mutant D222W/H/F/Y, S276F/Y, A224K/R/W, S272W/H, and T274W/H/F/Y, to enhance the p-p interaction, cation-p bond interaction, p-p interaction and the like of the sensitive region with a specific amino acid, so that identification of a fourth class of amino acids, including but not limited to W, P, F, Y, H, I, L and V, is achieved.

(V) Based on the large steric hindrance effect, the confined space of the current sensing region in the pore is further reduced, and the steric hindrance of the region is increased, such as mutant S276F/Y/I/L, S278F/Y/I/L/P, T274W/P, S236W and K238G/W/I/L/F/Y/P, to prolong the time of all amino acids passing through the region, enhance the current amplitude of ionic flow signals of small-volume amino acids, and enable the large-volume amino acids to generate a nearly fully blocked ion flow step, so that the volume of amino acids is specifically distinguished, and identification of a fifth class of small-volume amino acids, including but not limited to A, C, G, S, T and V, is achieved.

(VI) Based on the small steric hindrance effect, the stereostructure in the pore is regulated, the size of a key region is increased, such as mutant T218G/A, S276G/A, S278G/A, T274G/A, N226D/E and Q268S/T/G/A, and the electroosmotic flow in the nanopore is reduced based on the overall chargeability of the polypeptide, so that the current response of small-volume amino acids is further reduced, the current difference of large-volume amino acids is increased, and identification of a sixth class of large-volume amino acids, including but not limited to W, H, I, K, R and Y, is achieved.

(6) In the present invention, an amino acid which is easy to form hydrogen bonds is introduced into the inlet region of each of the orthogonal identification nanopores, and structure of the region in confined pore is adjusted, so that a secondary structure label region of a polypeptide is designed and constructed, in which amino acid residues inside the pore will have a hydrogen bond interaction with the polypeptides with different secondary structures, thereby inducing changes in specific ion flow blocking and specific ion mobility, and forming ion flow characteristics of the secondary structure label for calibrating and denoising an ion flow electrical signal of single protein sequencing during data processing.

(7) In the present invention, in view of amino acid identification errors possibly existing in the orthogonal amino acid identification, based on the previous research on ion flow migration tracks in the pore by the applicant team, ion flow confined perturbation techniques are adopted in combination with influences of specifically designed amplification temperature perturbation, alternating electric field perturbation and optical perturbation of the nanopore on the ion mobility in the pore to further identify ion migration frequency characteristics, thereby improving the amino acid identification capability at a nanopore measurement interface, and accurately obtaining sequence information of the single protein molecule.

(8) One or more measurements is made as the protein or polypeptide moves relative to the pore, specifically, a current passing through the pore is measured and analyzed, including characteristics such as amplitude, frequency, shape and duration of the current, thereby determining the presence or absence of one or more of the characteristics in the analyte; and characteristics of the current signal are resolved according to a mathematical transformation, and a database of polypeptides is created for mutual correction of data, thereby characterizing the protein or polypeptide.

The measurement time of the same class of polypeptide molecules in the 6 types of orthogonal identification nanopores shown in step (6) is different, and the retention time of different amino acids in the sensing sensitive region in each of the pores is different, so that the sequencing timelines of amino acids are different among the pores. Therefore, in the present invention, label amino acids are separately introduced in six types of amino acid identifications, such as histidine H in type (I), (II), (IV) and (VI) orthogonal identification nanopores, isoleucine (I) in type (III), (IV) and (VI) orthogonal identification nanopores, cysteine C in type (II), (III) and (V) orthogonal identification nanopores, and tyrosine Y in type (II), (IV) and (VI) orthogonal identification nanopores. Most of amino acids to be detected can be specifically identified in at least two types of orthogonal identification nanopores, so that the sequencing timelines in different nanopores are corrected and unified from multiple angles, and the intersection, correction and accurate integration of measured ion flow electrical signals of six nanopores are achieved.

In addition, in the present invention, post-translational modifications to amino acids can be detected at the same time as amino acid sequence determination. For example, phosphorylation modifications of serine (S), threonine (T), and histidine (H) can be identified in the type (I) and (II) orthogonal identification nanopores shown in step (6). Methylation modifications of aspartic acid (D) and glutamic acid (E) can be identified in the type (I) orthogonal identification nanopore shown in step (6). Glycosylation modifications of asparagine (N), threonine (T), and serine (S) can be identified in the type (I), (II) and (V) orthogonal identification nanopores shown in step (6). Therefore, in the present invention, it is expected to achieve accurate determination of the type, quantity and position of post-translational modifications of a specific amino acid while identifying the amino acid sequence of the polypeptide.

In the present invention, in the temperature perturbation of step (7) which significantly changes the random vibration of molecules, the interaction between the molecules and the like through temperature changes, the maximum stimulation to the degree of change of the ion mobility caused by interactions is achieved by accurately regulating the temperature of an experimental system in a range of 0-40° C., so that the signal-to-noise ratio of the frequency characteristic of a single amino acid is improved. In the alternating electric field perturbation with the frequency of 0.1-1 MHz, a functionalized perturbation system perturbation amplification nanopore is designed, the diameter and charge distribution of the pore are adjusted through site-directed mutagenesis, and ion-bound amino acids are introduced into the pore, such as mutant S236D/E/K/H/R, A260D/E/K/H/R, K238H/R/D/E, T240D/E, and S256H/R/W, such as Mg²⁺ and Ni⁺, so that changes of ion mobility caused by the interaction of amplified cations is induced under the specific alternating electric field frequency, and the identification of amino acids with small differences such as asparagine (N), glutamine (Q), isoleucine (I), and valine (V) is enhanced.

In the present invention, in the optical perturbation of step (7), a highly confined nanopore is designed to have various interaction types in the charge sensitive region, such as mutant S236W/H, K238I/L, S256Y/F/W, P249W and V250I/L/F/Y/W, so that weak interactions such as a hydrogen bond, a p-p interaction and a cation-p interaction of a polypeptide to be detected with a specific amino acid inside the pore are perturbed using infrared (10000-25000 nm) or ultraviolet (180-400 nm) light at a specific frequency to enhance identification of amino acids with similar weak interactions and isomers, such as serine (S) and threonine (T), and leucine (L) and isoleucine (I).

In the measurement of one, two, three, four, five or more characteristics of the protein or polypeptide of step (8), the one or more characteristics are preferably selected from: (i) the sequence of the protein or polypeptide; (ii) whether a protein or polypeptide being modified and the type, position and number of amino acids modified; (iii) the length of the protein or polypeptide; (iv) the consistency of the protein or polypeptide; (v) the conformation of the protein or polypeptide; (vi) the secondary structure of a protein or polypeptide.

### BRIEF DESCRIPTION OF THE DRAWINGS

FIG. 1: a flow chart of the method for sequencing a protein/polypeptide.

FIG. 2: original current tracks of two tripeptide molecules, Glu-Gly-Cys and Glu-Cys-Gly, as detected by an N226Q charge screening Aerolysin nanopore.

FIG. 3: an original current track of a molecular mixture of two tripeptides, Glu-Gly-Cys and Glu-Cys-Gly, as detected by a T232K charge screening Aerolysin nanopore.

FIG. 4: original current tracks of two tripeptide molecules, Glu-Gly-Cys and Glu-Cys-Gly, as detected by a T232K/K238Q polypeptide sequencing Aerolysin nanopore.

FIG. 5: original current tracks of a template polypeptide molecule and two phosphorylated polypeptide molecules as detected by a wild-type charge screening Aerolysin nanopore.

FIG. 6: original current tracks of a template polypeptide molecule and two phosphorylated polypeptide molecules as detected by a T232K/K238Q phosphorylation detection Aerolysin nanopore.

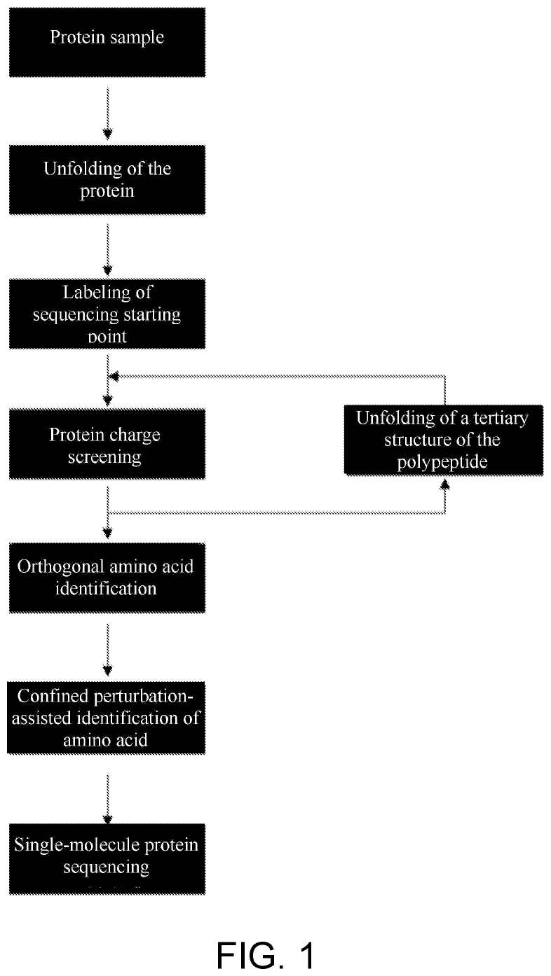

### DESCRIPTION OF THE EMBODIMENTS

### Example 1

Provided was a method for sequencing polypeptide molecules by a cysteine-specific Aerolysin nanopore, in which Glu was taken as a guide chain for a polypeptide, and amino acid sequences of two polypeptide molecules were Glu-Gly-Cys and Glu-Cys-Gly, respectively. The specific steps are as follows:

(1) N226Q and T232K protein charge screening nanopore were designed, and corresponding mutant Proerolysin proteins were expressed and purified for pore construction by site-directed mutagenesis, with the specific steps referred to the patent CN202010131704.8.

(2) 1 mg/mL Proerolysin protein was mixed with trypsin in a 10:1 ratio, and incubated at room temperature for 6 h to obtain Aerolysin monomeric protein with pore-forming activity.

(3) The experiment temperature was controlled at 22±1° C. 1 mL of buffer solution (1.0 M KCl, 10 mM Tris, 1.0 mM EDTA, pH=8) was added to each of the two detection cells, and a phospholipid bilayer was prepared by the Czochralski method, with the specific steps referred to the patent CN201510047662.9.

(4) After a stable phospholipid bilayer was formed, 200 mV voltage was applied and 1 μL of Aerolysin monomeric protein was added into the cis detection cell. Aerolysin monomers were self-assembled to form a heptamer and inserted into the phospholipid membrane to form a stable nanopore, and simultaneously, the ion flow took a jump, so that a stable open-pore current was obtained under the voltage of 100 mV.

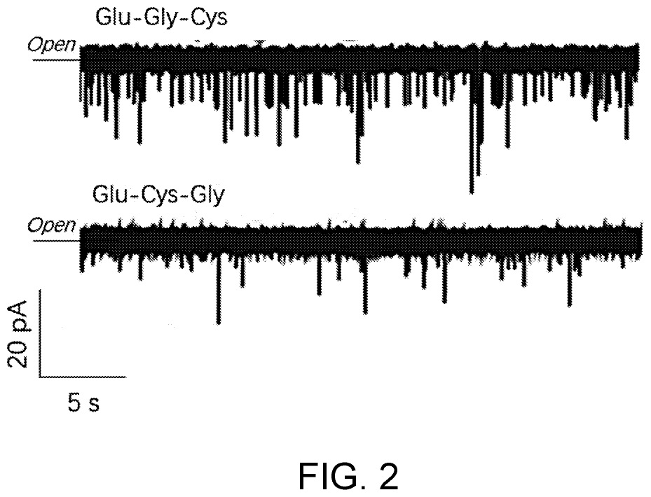

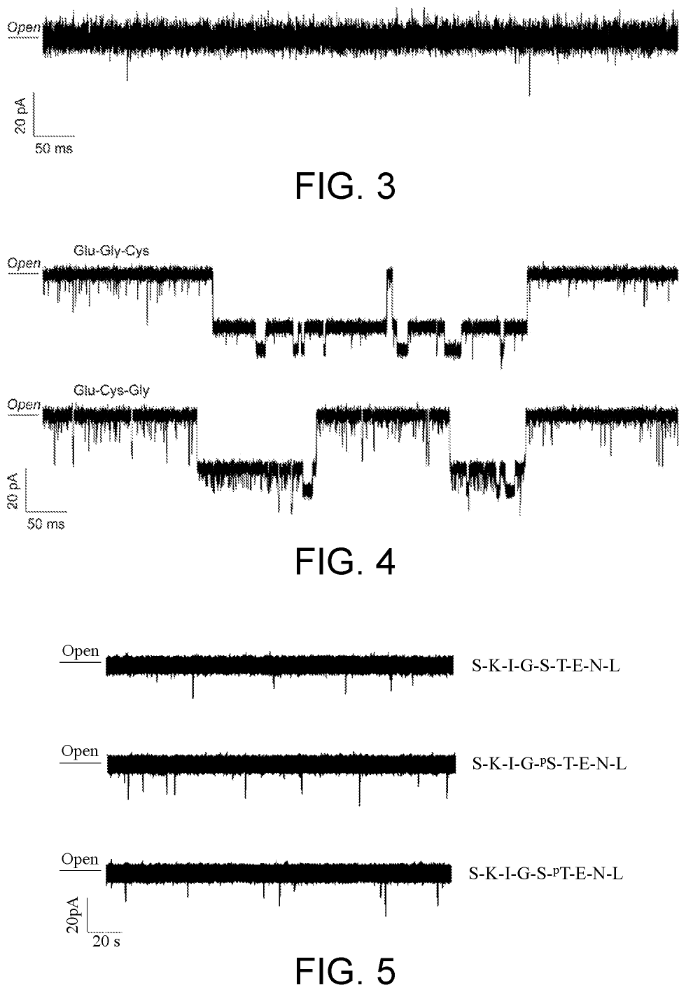

(5) 44 of 50 mM tripeptide chain was added into the cis detection cell, and an external voltage of 120 mV was applied. Original current tracks acquired are shown in FIGS. 2-3. FIGS. 2 and 3 show original current tracks of two tripeptide molecules, Glu-Gly-Cys and Glu-Cys-Gly, as detected by N226Q and T232K preliminary chargeability screening Aerolysin nanopores, respectively. Firstly, the chargeability of the polypeptide was determined from the preliminary chargeability screening pores, and the two tripeptide molecules both showed current blocked signals in the N226Q preliminary chargeability screening pore (FIG. 2). There were no current blocked signals in the T232K preliminary chargeability screening pore. The current track of a mixture of two polypeptide molecules added is shown in FIG. 3. Therefore, it is determined that the two polypeptide molecules are both driven by negative charges.

(6) A T232K/K238Q double-mutant polypeptide sequencing pore was designed, and mutant Proerolysin protein was expressed and purified for pore construction by site-directed mutagenesis.

(7) The nanopore construction steps were repeated, and two polypeptide molecules were added into the cis detection cell, respectively. As shown in FIG. 4, the two polypeptide molecules generated two blocked signals in the T232K/K238Q double-mutant polypeptide sequencing pore, wherein the characteristic double-step blocked signal with longer blocking time was the blocked signal generated by the polypeptide molecule dragged into the pore starting from the N terminus by the guide chain Glu, and the signal with shorter blocking time was the blocked signal generated by the polypeptide molecule not dragged into the pore by the guide chain. The sequences of the two polypeptide molecules can be distinguished according to the shape, blocking time and blocking degree of the blocked signals.

### Example 2

Provided was a method for detecting phosphorylated polypeptides using mutant Aerolysin nanopores, in which S-K-I-G was used as a guide chain, the sequence of a template polypeptide was S-K-I-G-S-T-E-N-L, and the sequences obtained by phosphorylation modification of serine at the fifth position and threonine at the sixth position were S-K-I-G-^{P}S-T-E-N-L and S-K-I-G-S-^{P}T-E-N-L, respectively. The specific steps are as follows:

(1) A wild-type preliminary chargeability screening nanopore was designed, and a wild-type Proerolysin protein was expressed and purified for pore construction, with the specific steps referred to the patent CN202010131704.8.

(2) 1 mg/mL Proerolysin protein was mixed with trypsin in a 10:1 ratio, and incubated at room temperature for 6 h to obtain Aerolysin monomeric protein with pore-forming activity.

(3) The experiment temperature was controlled at 22±1° C. 1 mL of buffer solution (1.0 M KCl, 10 mM Tris, 1.0 mM EDTA, pH=8) was added to each of the two detection cells, and a phospholipid bilayer was prepared by the Czochralski method, with the specific steps referred to the patent CN201510047662.9.

(4) After a stable phospholipid bilayer was formed, 200 mV voltage was applied and 1 μL of Aerolysin monomeric protein was added into the cis detection cell. Aerolysin monomers were self-assembled to form a heptamer and inserted into the phospholipid membrane to form a stable nanopore, and simultaneously, the ion flow took a jump, so that a stable open-pore current was obtained.

(5) 5 μL of 1 mM polypeptide solution was added into the cis detection cell, and an external voltage of 100 mV was applied. The acquired original current tracks are shown in FIG. 5. Three polypeptide molecules generated a few current blocked signals in the wild-type Aerolysin nanopore, therefore, it is determined that the three polypeptide molecules are driven by negative charges.

(6) A T232K/K238Q double-mutant phosphorylation detection pore was designed, and mutant Proerolysin protein was expressed and purified for pore construction by site-directed mutagenesis.

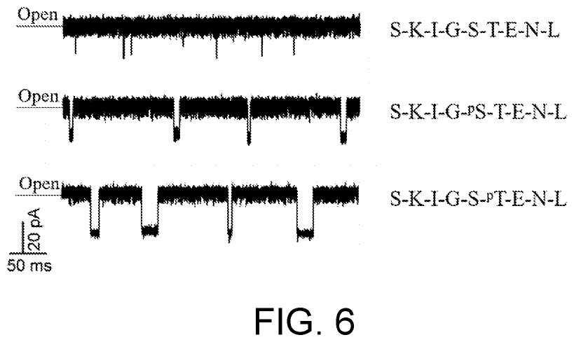

(7) The nanopore construction steps were repeated, and three polypeptide molecules were added into the cis detection cell, respectively. As shown in FIG. 6, the template polypeptide molecule and the two phosphorylated polypeptide molecules can be distinguished according to the shape, blocking time and blocking degree of blocked signals generated by the polypeptide molecules in the T232K/K238Q pore.

The general principles, principal features, and advantages of the present invention are revealed and described in the above examples. It should be understood by those skilled in the art that the present invention is not limited to the above examples, which are merely illustrative of the principles of the present invention. Various changes and modifications may be made without departing from the spirit and scope of the present invention, and those changes and modifications fall into the claimed scope of the present invention. The claimed scope of the present invention is defined by the appended claims and the equivalents thereof.

1. A method for sequencing a protein/polypeptide using Aerolysin nanopores, comprising the following steps:
(1) unfolding of the protein; (2) terminus labeling of protein sequencing; (3) protein charge screening; (4) unfolding of a tertiary structure of the polypeptides; (5) orthogonal identification of amino acids; (6) confined perturbation-assisted identification of amino acids; and (7) single-molecule protein sequencing.

2. The method for sequencing a protein/polypeptide using Aerolysin nanopores according to claim 1, wherein in the unfolding of the protein of step (1), a single protein molecule must have its high order structure unraveled to enter a single nanopore in a linear chain form before the sequencing based on nanopores, and the single protein will be unfolded by a temperature and pH regulation method.

3. The method for sequencing a protein/polypeptide using Aerolysin nanopores according to claim 1, wherein in the terminus labeling of protein sequencing of step (2), the N-terminus or C-terminus of an unfolded polypeptide chain is labeled with a peptide nucleic acid, an oligonucleotide, a polypeptide chain or an organic functional group having a specific sequence as a sequencing origin to obtain an label signal of ion flow starting point.

4. The method for sequencing a protein/polypeptide using Aerolysin nanopores according to claim 1, wherein in the protein charge screening of step (3), protein charge screening nanopores are designed.

5. The method for sequencing a protein/polypeptide using Aerolysin nanopores according to claim 1, wherein in the unfolding of a tertiary structure of the polypeptide of step (4), a tertiary structure unfolding nanopore is further designed for assisting the protein charge screening, that is, an unfolding region is constructed at an inlet of a biological nanopore to further open a molecular structure of the polypeptide, i.e., mutant T284/F/Y/I/L/W or G214/F/Y/I/L/W.

6. The method for sequencing a protein/polypeptide using Aerolysin nanopores according to claim 1, wherein in the orthogonal identification of amino acids of step (5), aiming at sequencing of linear polypeptide molecules for each chargeability, nanopores at least comprising the following six types of orthogonal nanopores are designed:
(a) identification of a first class of amino acids, comprising but not limited to H, R, K, E, D, Q, N and W, intended to be achieved based on an electrostatic interaction, i.e., mutant T218K/R/H/D/E, S278K/R/H/D/E, S276K/R/H/D/E, T274K/R/H/D/E or A224Q/N/D/E/R/K/H;
(b) identification of a second class of amino acids, comprising but not limited to Q, N, Y, T, S, C, G and H, intended to be achieved based on hydrogen bond and hydrophilic interactions, i.e., mutant T218N/Q, Q212R/K/H, D209S/T, S276Q/N, D222G/A/S or A224E/D;
(c) identification of a third class of amino acids, comprising but not limited to I, L, M, V, P, A, C and G, intended to be achieved based on a van der Waals interaction, i.e., mutant R220S/T/A, D222G/A, S236I/L/V, G270I/L, T232I/L/V, T274G/A/I/L or K238F/Y/W;
(d) identification of a fourth class of amino acids, comprising but not limited to W, P, F, Y, H, I, L and V, intended to be achieved based on a large p bond in side chains of part of the amino acids, i.e., mutant D222W/H/F/Y, S276F/Y, A224K/R/W, S272W/H or T274W/H/F/Y;
(e) identification of a fifth class of small-volume amino acids, comprising but not limited to A, C, G, S, T and V, intended to be achieved based on a small steric hindrance effect, i.e., mutant S276F/Y/I/L, S278F/Y/I/L/P, T274W/P, S236W or K238G/W/I/L/F/Y/P; and
(f) identification of a sixth class of large-volume amino acids, comprising but not limited to W, H, I, K, R and Y, intended to be achieved based on a large steric hindrance effect, i.e., mutant T218G/A, S276G/A, S278G/A, T274G/A, N226D/E or Q268S/T/G/A.

7. The method for sequencing a protein/polypeptide using Aerolysin nanopores according to claim 1, wherein in the confined perturbation-assisted identification of amino acids of step (6), in view of identification errors possibly introduced between part of amino acids with small structural differences and isomer amino acids, alternating electric field and optical perturbation measurement systems are introduced and a perturbation amplification nanopore for the perturbation systems is designed to further improve sequencing accuracy in combination with a specific nanopore, wherein the specific nanopore is shown as follows:
(a) mutant S236D/E/K/H/R, A260D/E/K/H/R, K238H/R/D/E, T240D/E or S256H/R/W in combination with the alternating electric field perturbation system; and
(b) mutant S236W/H, K238I/L, S256Y/F/W, P249W or V250I/L/F/Y/W in combination with the optical perturbation system.

8. The method for sequencing a protein/polypeptide using Aerolysin nanopores according to claim 1, wherein the single-molecule protein sequencing of step (7) is performed by:
(a) bringing the protein or polypeptide into a contact with the pore, so that the protein or polypeptide moves relative to the pore; and
(b) measuring an ion current passing through the pore as the protein or polypeptide moves relative to the pore, wherein the current is indicative of one or more characteristics of the protein or polypeptide, and comprises shape, amplitude and duration of a current signal, resolving characteristics of the current signal according to a mathematical transformation, and creating a database of polypeptides for mutual correction of data, thereby characterizing the protein or polypeptide.

9. The method for sequencing a protein/polypeptide using Aerolysin nanopores according to claim 1, comprising the following specific steps:
sample pretreatment: breaking internal hydrogen bonds of the protein by raising the temperature to 60-100° C. and decreasing the pH of a solution to 0-5, and breaking S—S bonds of the protein using a reducing agent tri(2-carboxyethyl)phosphine (TCEP) or dithiothreitol (DTT) at the same time, so that polypeptide chains in the single protein are released and linearized;
specifically modifying the N-terminus of the polypeptide chain with a peptide nucleic acid PNA, an oligonucleotide, a polypeptide chain or an organic functional group, so that a specific ion flow blocked signal or fluorescent signal is generated at the beginning or the end of the polypeptide entering the nanopore, thereby determining the starting point of sequencing of the single polypeptide molecule in the nanopore, and providing a starting time label for mutual correction of parallel sequencing signals of a plurality of orthogonal identification nanopores;
using a denaturant and designing and constructing a “tertiary structure unfolding nanopore” to achieve the unfolding of the tertiary structure of the polypeptide, wherein the “tertiary structure unfolding nanopore” is designed as follows: bionically constructing a central amino acid environment of a proteasome 19S domain at the inlet of the Aerolysin nanopore to enhance a specific non-covalent interaction between the polypeptide and the nanopore, and gradually destroying weak interactions inside the polypeptide molecule by virtue of electric driving forces to drive the polypeptide molecule to enter a confined pore and achieve linear unfolding, so that the great challenge of the tertiary structure of the polypeptide on sequencing of the polypeptide in the nanopore is overcome;
designing functionalized Aerolysin nanopores capable of driving polypeptides with different chargeabilities, and preliminarily screening the chargeabilities of the polypeptides to match the selection of orthogonal sequencing nanopores in the next step;
constructing 6 types of orthogonal identification nanopores for specifically identifying amino acid sequences of polypeptides for each chargeability based on an electrostatic interaction, hydrogen bond and hydrophilic interactions, a van der Waals interaction, a large p-bond interaction of amino acids, a large steric hindrance effect and a small steric hindrance effect;
introducing amino acids which are easy to form a hydrogen bond into the inlet region of each orthogonal identification nanopore, and adjusting the confined pore structure of the region, thereby designing and constructing a secondary structure label region of polypeptides, in which amino acid residues inside the pore will have a hydrogen bond interaction with the polypeptides with different secondary structures, thereby inducing changes in specific ion flow blocking and specific ion mobility, and forming ion flow characteristics of the secondary structure label for calibrating and denoising an ion flow electrical signal of single protein sequencing during data processing;
in view of amino acid identification errors possibly existing in the orthogonal amino acid identification, further identifying ion migration frequency characteristics by adopting an ion flow confined perturbation technique in combination with influences of specifically designed amplification temperature perturbation, alternating electric field perturbation and optical perturbation of the nanopore on the ion mobility in the pore, thereby improving the amino acid identification capability at a nanopore measurement interface, and accurately obtaining sequence information of the single protein molecule; and
making one or more measurements as the protein or polypeptide moves relative to the Aerolysin nanopore, specifically, measuring and analyzing a current passing through the pore, comprising characteristics such as amplitude, frequency, shape and duration of the current, thereby determining the presence or absence of one or more of the characteristics in the analyte; and resolving characteristics of the current signal according to a mathematical transformation, and creating a database of polypeptides for mutual correction of data, thereby characterizing the protein or polypeptide.

10. The method for sequencing a protein/polypeptide using Aerolysin nanopores according to claim 1, wherein the organic functional group in step (2) is FAM, VIC, CY5, HEX or ROX.

11. The method for sequencing a protein/polypeptide using Aerolysin nanopores according to claim 1, wherein the denaturant used in step (3) is guanidine hydrochloride or GdHCl.

12. The method for sequencing a protein/polypeptide using Aerolysin nanopores according to claim 1, wherein the electric driving forces in step (3) are an electrophoresis force, an electroosmotic flow, and a dielectrophoresis force.

13. The method for sequencing a protein/polypeptide using Aerolysin nanopores according to claim 1, wherein the designing functionalized Aerolysin nanopores capable of driving polypeptides with different chargeabilities in step (3) comprises: adopting 4 “protein charge screening nanopores” for specifically capturing 4 types of polypeptides with chargeabilities, namely negatively charged polypeptides, positively charged polypeptides, electrically neutral polypeptides with positive and negative charges shielded from each other, and electrically neutral polypeptides with positive and negative charges separated, respectively.

14. The method for sequencing a protein/polypeptide using Aerolysin nanopores according to claim 5, wherein the 4 “protein charge screening nanopores” are a “protein charge screening nanopore” for specifically identifying the negatively charged polypeptides, a “protein charge screening nanopore” for specifically identifying the positively charged polypeptides, a “protein charge screening nanopore” for specifically identifying the electrically neutral polypeptides with positive and negative charges shielded from each other, and a “protein charge screening nanopore” for specifically identifying the electrically neutral polypeptides with positive and negative charges separated, respectively; and the 4 nanopores are designed as follows:
(i) the “protein charge screening nanopore” for specifically identifying the negatively charged polypeptides: by adjusting the diameter of the key region inside the Aerolysin nanopore or transferring charges in the pore to a region of a larger diameter, that is, constructing mutant T274N/Q/I/L, T232D/E, K238H/D/R/F/A/C/G/Q/E/K/L/M/N/S/Y/T/I/W/P/V or S280T/N/Q/H/I/L, the electroosmotic flow inside the nanopore that is determined by the charge at the narrowest part of the pore is controlled to be zero, and the dielectrophoresis force is reduced, so that a single negatively charged polypeptide is driven into the nanopore by the electrophoresis force;
(ii) the “protein charge screening nanopore” for specifically identifying the positively charged polypeptides: by introducing or increasing the distribution of negative charges in the Aerolysin nanopore, that is, constructing mutant T274D/E, T218D/E, S276D/E, S278D/E, K238A/N/D/E/Q, R282D/E/S/T/N/Q/A or R220D/E/S/T/N/Q/A, the electroosmotic flow determined by cations inside the pore is adjusted; in the experiment, a reverse voltage is applied to achieve efficient capture of a single positively charged polypeptide;
(iii) the “protein charge screening nanopore” for specifically identifying the electrically neutral polypeptides with positive and negative charges shielded from each other: for the electrically neutral polypeptide with positive and negative charges shielded from each other, by introducing positively charged amino acids into a region of a smaller diameter of the Aerolysin nanopore, that is, constructing mutant T218K/R/H/N/Q, S276K/R/H, S278K/R/H/N/Q, S274K/R/H, N226K/R/H, S272K/R/H, G270K/R/H, S228K/R/H, Q268K/R/H, T230K/R/H, A266K/R/H, T232K/R/H, S264K/R/H, G234K/R/H, N262K/R/H, S236K/R/H, A260K/R/H or S280N/Q, the electroosmotic flow determined by anions inside the pore is enhanced, so that the capture efficiency of the nanopore to the electrically neutral polypeptide with positive and negative charges shielded from each other is enhanced, and a specific ion flow response is obtained; and
(iv) the “protein charge screening nanopore” for specifically identifying the electrically neutral polypeptides with positive and negative charges separated: for the electrically neutral polypeptides with positive and negative charges separated, by enhancing the potential gradient at the inlet of the Aerolysin nanopore, that is, constructing mutant S280Q/N/A, T284Q/N/A or G214Q/N/A, the non-linear electric field strength is adjusted, so that a single electrically neutral polypeptide with positive and negative charges separated is driven into the pore by the dielectrophoretic force.

15. The method for sequencing a protein/polypeptide using Aerolysin nanopores according to claim 5, wherein the 6 types of orthogonal identification nanopores of step (5) are constructed as follows:
(I) based on the electrostatic interaction, charged amino acids are introduced into an existing current sensing region in the pore, that is, constructing mutant T218K/R/H/D/E, S278K/R/H/D/E, S276K/R/H/D/E, T274K/R/H/D/E or A224Q/N/D/E/R/K/H; the introduction of the charged amino acids can enhance hydrogen bond, salt bond and cation-p interactions between the pore and the amino acids to be sequenced, so that identification of a first class of amino acids, comprising but not limited to H, R, K, E, D, Q, N and W, is achieved;
(II) based on the hydrogen bond and hydrophilic interactions, the potential gradient of a current sensing region in the pore is regulated, that is, constructing mutant T218N/Q, Q212R/K/H, D209S/T, S276Q/N, D222G/A/S or A224E/D, to increase the speed of charged amino acids passing through the region and prolong the retention time of polar uncharged amino acids in the region, so that identification of a second class of amino acids, comprising but not limited to Q, N, Y, T, S, C, G, and H, is achieved; wherein the histidine His has an R group with pKa of about 7, and can be made uncharged through fine adjustment of pH, thus enabling characteristic differentiation in a specific nanopore based on its hydrogen bond interaction with a key sensing region of the nanopore;
(III) based on the van der Waals interaction, the overall potential distribution and the stereostructure distribution of the pore are regulated, and hydrophobic amino acids are introduced, that is, constructing mutant R220S/T/A, D222G/A, S236I/L/V, G270I/L, T232I/L/V, T274G/A/I/L or K238F/Y/W, to transfer the current sensing region from a electrostatic sensitive region of a wild-type pore to a hydrophobic region of a mutant pore, and prolong the retention time of a specific amino acid in the region through the interaction to obtain a characteristic ion flow signal, so that identification of a third class of amino acids, comprising but not limited to I, L, M, V, P, A, C and G, is achieved;
(IV) based on the large p-bond interaction of part of amino acids, the composition of a current sensing region in the Aerolysin nanopore is reconstituted on the basis of regulating the stereostructure and potential distribution of the pore, and a sensitive region with positively charged amino acids and hydrophobic amino acids predominated is constructed, that is, constructing mutant D222W/H/F/Y, S276F/Y, A224K/R/W, S272W/H or T274W/H/F/Y, to enhance the p-p interaction, cation-p bond interaction, and p-p interaction of the sensitive region with a specific amino acid, so that identification of a fourth class of amino acids, comprising but not limited to W, P, F, Y, H, I, L and V, is achieved;
(V) based on the large steric hindrance effect, the confined space of the current sensing region in the pore is further reduced, and the steric hindrance of the region is increased, that is, constructing mutant S276F/Y/I/L, S278F/Y/I/L/P, T274W/P, S236W or K238G/W/I/L/F/Y/P, to prolong the time of all amino acids passing through the region, enhance the current amplitude of ionic flow signals of small-volume amino acids, and enable the large-volume amino acids to generate a nearly fully blocked ion flow step, so that the volume of amino acids is specifically distinguished, and identification of a fifth class of small-volume amino acids, comprising but not limited to A, C, G, S, T and V, is achieved; and
(VI) based on the small steric hindrance effect, the stereostructure in the pore is regulated, the size of a key current region is increased, that is, constructing mutant T218G/A, S276G/A, S278G/A, T274G/A, N226D/E or Q268S/T/G/A, and the electroosmotic flow in the nanopore is reduced based on the overall chargeability of the polypeptide, so that the current response of small-volume amino acids is further reduced, the current difference of large-volume amino acids is increased, and identification of a sixth class of large-volume amino acids, comprising but not limited to W, H, I, K, R and Y, is achieved.
